(19) United States
(12) Patent Application Publication
(10) Pub. No.: US 2021/0016264 A1
(43) Pub. Date: Jan. 21, 2021

(54) CATALYTIC TEST PAPER PREPARED BY COMPOSITING METAL PARTICLE-EMBEDDED BACTERIAL CELLULOSE WITH PLANT FIBERS, AND METHOD THEREFOR

(71) Applicant: SOUTH CHINA UNIVERSITY OF TECHNOLOGY, Guangdong (CN)

(72) Inventors: Zhouyang XIANG, Guangdong (CN); Fachuang LV, Guangdong (CN); Jun LI, Guangdong (CN)

(73) Assignee: SOUTH CHINA UNIVERSITY OF TECHNOLOGY, Guangdong (CN)

(21) Appl. No.: 16/982,055

(22) PCT Filed: Oct. 31, 2018
(86) PCT No.: PCT/CN2018/113233

§ 371 Date: Sep. 18, 2020

(30) Foreign Application Priority Data
Mar. 19, 2018 (CN) 201810224440.3

(51) Int. Cl. B01J 31/06; B01J 23/44; B01J 23/755; B01J 23/50; B01J 35/06; B01J 37/02; B01J 37/16; B01J 37/06

(52) U.S. Cl. CPC B01J 31/061; B01J 2531/005; B01J 23/755; B01J 23/50; B01J 35/06; B01J 37/0217; B01J 37/0228; B01J 37/16; B01J 37/0236; B01J 37/06; B01J 2231/4211; B01J 2231/4261; B01J 2231/4266; B01J 2231/005; B01J 23/44

(57) ABSTRACT

Disclosed is a catalytic test paper prepared by compositing metal particle-embedded bacterial cellulose with plant fibers, and a preparation method therefor. Hydroxyl groups of bacterial cellulose are bonded with a nitrogen-containing or phosphorus-containing organic small molecule compound. By means of a chelation between a nitrogen or phosphorus atom with a metal, transition metal ions are adsorbed to a nanoporous surface of bacterial cellulose, and the transition metal ions are reduced in situ to obtain bacterial cellulose embedded with metal nanoparticles. The bacterial cellulose is composited with the plant fiber, and the catalytic test paper is prepared by a papermaking method. The catalytic test paper has the advantages of convenient use and recovery, high reusability, simple design, low manufacturing cost, higher catalytic efficiency, a green degradable support material, etc.

### TECHNICAL FIELD

The present invention relates to the fields of papermaking technology and organic catalysis, and more particularly, to a catalytic test paper prepared by compositing metal particle-embedded bacterial cellulose with plant fibers, and a method therefor.

### BACKGROUND

Metal nanoparticles in a heterogeneous catalyst have a catalytic reaction efficiency close to a molecular level due to a small size and a large specific surface area thereof. However, it is still a problem to effectively separate and recover the metal nanoparticles, with a time-consuming and labor-intensive process and a high cost. In addition, due to high surface energy and Van der Waals' force, the metal nanoparticles are easy to aggregate, thus reducing a catalytic activity thereof. It is a common problem to be solved to support the metal nanoparticles on a suitable carrier to improve their stability, separability and recoverability thereof, so that the metal nanoparticles have the advantages of both homogeneous and heterogeneous catalysts. Some commonly used carriers for supporting a metal nano-catalyst include: mesoporous organosilicon and grafted silicon foam, layered double hydroxide, clay, zeolite molecular sieve, various metal oxides, active or nitrogen-doped carbon, polymer network, graphene, resin, etc. However, most of these carriers may easily leach or degrade the catalyst through reuse, thus affecting a reaction yield and limiting the reuse. In addition, most of the carriers have a low specific surface area, so that it is difficult to endow the catalyst and a reactant with a large contact area. Therefore, it is still a major technical problem to be solved to find a suitable metal nanoparticle carrier, which is able to endow the catalyst with a high catalytic efficiency and strong recoverability and reusability at the same time.

Bacterial cellulose is a new bio-based material, which is secreted and compounded by microorganisms, and has extremely high cellulose purity and crystallinity, and a fine microscopic network structure. Under microscopic morphology, uniformly separated and interwoven cellulose microfibril structures (with a diameter of 2 nm to 100 nm) endow the bacterial cellulose with an ultra-fine three-dimensional network structure. These interwoven microfibril structures form a large number of nano-scale tunnels and porous structures on a surface of the bacterial cellulose, and have a potential to lock and disperse the metal nanoparticles, and prevent the metal nanoparticles from aggregation. However, it is costly to use pure bacterial cellulose as the carrier of the metal nanoparticles, with a poor mechanical stiffness and a weak liquid permeability, thus weakening contact between the reactant and the metal particles.

Bacterial cellulose is able to be closely bound with plant fibers through a large number of free hydroxyl groups due to a fine network structure thereof. Therefore, paper materials can be endowed with a special function through physically and chemically modifying the bacterial cellulose with special functional characteristics. Therefore, an innovation point of the present invention lies in preparing a catalytic test paper by compositing metal particle-loaded bacterial cellulose with plant fibers. In the catalytic test paper, the bacterial cellulose ensures good dispersion and stability of the metal nanoparticles, while the plant fiber is used as a matrix to ensure a mechanical stiffness of the catalyst carrier and a permeability of the reactant. The catalytic test paper has the advantages of extremely convenient use and recovery, a high reusability, a simple design, a low manufacturing cost, a high catalytic efficiency, a green degradable support material, etc.

### SUMMARY

In order to find a green degradable metal nanoparticle carrier and endow a catalyst with a high catalytic efficiency, and strong recoverability and reusability at the same time, an objective of the present invention is to provide a method for preparing a catalytic test paper by compositing metal particle-embedded bacterial cellulose with plant fibers. In the catalytic test paper, the bacterial cellulose ensures good dispersion and stability of the metal nanoparticles, while the plant fiber is used as a matrix to ensure a mechanical stiffness of the catalyst carrier and a permeability of a reactant. The catalytic test paper has the advantages of extremely convenient use and recovery, a high reusability, a simple design, a low manufacturing cost, a high catalytic efficiency, a green degradable support material, etc.

The objective of the present invention is achieved by the following technical solutions.

A method for preparing a catalytic test paper by compositing metal particle-embedded bacterial cellulose with plant fibers includes the following steps:

(1) chemically bonding a nitrogen-containing or phosphorus-containing organic small molecule compound with a large number of hydroxyl groups in a structure of bacterial cellulose to obtain a functionalized bacterial cellulose having a nitrogen-containing or phosphorus-containing group, wherein the beneficial effect thereof is that through chelation between the nitrogen-containing or phosphorus-containing group and metal atoms, a binding stability between metal nanoparticles and the bacterial cellulose is strengthened, and a reusability of the catalytic test paper is improved;

(2) preparing an aqueous solution of an inorganic salt of a transition metal, adding the aqueous solution into the functionalized bacterial cellulose prepared in the step (1), with the reaction may be performed with heating according to a solubility of the inorganic salt of the transition metal, stirring and reacting for more than 3 hours until a nitrogen-containing or phosphorus-containing functional group adsorbs transition metal ions onto a nanoporous surface of the bacterial cellulose till saturation, separating and washing with deionized water, wherein the beneficial effect thereof is that dispersion of metal particles on the surface of the bacterial cellulose is strengthened and a catalytic efficiency is improved;

(3) reducing the transition metal ions adsorbed on the surface of the bacterial cellulose in the step (2) in situ to obtain bacterial cellulose embedded with transition metal nanoparticles, wherein the beneficial effect thereof is that the dispersion of the metal particles on the surface of the bacterial cellulose is strengthened, and the catalytic efficiency is improved; and

(4) mixing a plant fiber pulp with the bacterial cellulose embedded with the transition metal nanoparticles prepared in the step (3), uniformly dispersing the mixed pulp by a standard paper pulp disintegrator, manufacturing the mixed pulp into a paper sheet, and preparing the paper sheet into the catalytic test paper; and drying the test paper to an equilibrium weight, and keeping the test paper away from light and air, wherein the beneficial effect thereof is that a mechanical strength of a catalyst carrier (the catalytic test paper) may be ensured by using the plant fiber as a matrix, and due to a porosity of the plant fiber, a permeability of a reactant of the carrier can be improved, and a contact probability between the reactant and the metal particles can be increased, thus improving the catalytic efficiency.

Further, the bacterial cellulose is secreted in vitro by a bacterial microorganism, such as one of gluconacetobacter, acetobacter, agrobacterium, pseudomonas, achromobacter, alcaligenes, aerobacter, azotobacter, rhizobium and sarcina, and a culturing condition is a static or dynamic fermentation culturing condition.

Furthermore, the nitrogen-containing or phosphorus-containing organic small molecule compound in the step (1) includes (but is not limited to) ethylenediamine, tetraethylenepentamine, diethylenetriamine, polyethyleneimine, N-methylimidazole, chlorodiphenyl phosphine, etc.

Further, the transition metal in the step (2) includes (but is not limited to) palladium, chromium, nickel, silver, copper, gold and other metals with a catalytic property.

Further, a method for bonding the nitrogen-containing or phosphorus-containing organic small molecule with the hydroxyl groups of the bacterial cellulose in the step (1) includes (but is not limited to) oxidizing the hydroxyl groups on C2 and C3 bond positions of the bacterial cellulose into aldehyde groups in water by using an oxidant, such as a periodate, a 2,2,6,6-tetramethylpiperidine-1-oxyl radical (TEMPO) agent, etc., and then bonding the aldehyde groups with the nitrogen-containing compound through a reductive amination reaction.

Further, a method for bonding the nitrogen-containing or phosphorus-containing organic small molecule with the hydroxyl groups of the bacterial cellulose in the step (1) includes (but is not limited to) reacting the bacterial cellulose with a halogen-containing epoxy compound such as epichlorohydrin in concentrated alkaline (with a mass fraction of 10% to 20%), bonding epoxy groups with the hydroxyl groups of the bacterial cellulose, and then reacting the epoxy groups with the nitrogen-containing organic small molecule compound.

Further, a method for bonding the nitrogen-containing or phosphorus-containing organic small molecule with the hydroxyl groups of the bacterial cellulose in the step (1) includes (but is not limited to) reacting the bacterial cellulose with thionyl chloride, etc. under dimethylformamide (DMF) or N,N-dimethylacetamide (DMAc), bonding chlorine atoms to the hydroxyl groups of the bacterial cellulose, and then reacting with the nitrogen-containing organic small molecular compound.

Further, a method for bonding the nitrogen-containing or phosphorus-containing organic small molecule with the hydroxyl groups of the bacterial cellulose in the step (1) includes (but is not limited to) bonding with the hydroxyl groups of the bacterial cellulose by using chlorodiphenyl phosphine, etc. in a condition that pyridine is used as a solvent.

Further, a method for reducing the transition metal ions adsorbed by the bacterial cellulose in situ in the step (3) includes (but is not limited to) soaking the bacterial cellulose adsorbed with the transition metal ions in a solution of sodium borohydride, sodium cyanoborohydride, hydroxylamine hydrochloride and other reducing agents.

Further, the plant fiber pulp in the step (4) is a papermaking raw material prepared from a wood fiber, a non-wood plant fiber or a secondary fiber by a mechanical or chemical pulping method.

Further, a drying temperature in the step (4) is about 110° C.

Further, a consistency of the disintegrated pulp in the step (4) is 1 wt %.

The catalytic test paper is prepared by the above method. The catalytic test paper may be placed in a nylon net frame during use, and then added into a reaction medium which needs a catalytic reaction, and reaction efficiency can be improved by magnetic stirring or other means, wherein the beneficial effect thereof is that the catalytic test paper is protected and the reusability is improved.

Further, the catalytic reaction includes (but is not limited to) a Suzuki-Miyaura coupling reaction, a Heck reaction, a Sonogashira reaction, degradation of a nitro-aromatic compound, etc.

Compared with the prior art, the present invention has the following advantages.

1. In the catalytic test paper prepared according to the present invention, the bacterial cellulose ensures good dispersion and stability of the metal nanoparticles, while the plant fiber is used as the matrix to ensure the mechanical stiffness of the catalyst carrier and the permeability of the reaction medium.

2. The catalytic test paper prepared according to the present invention has the advantages of extremely convenient use and recovery, a high reusability, a simple design, a low manufacturing cost, a high catalytic efficiency, a green degradable support material, etc.

### BRIEF DESCRIPTION OF THE DRAWINGS

FIG. 1 is a flow chart of a method for preparing a catalytic test paper by compositing metal particle-embedded bacterial cellulose with plant fibers.

### DETAILED DESCRIPTION

The present invention is further described in detail through the embodiments hereinafter, but the implementation of the present invention is not limited to the embodiments.

Bacterial cellulose in the embodiment was secreted by Glucoacetobacter xylinus. Main components of a bacterial culturing medium included 50 mL of fermented coconut water, 0.1 g of ammonium sulfate, 0.1 g of magnesium sulfate, 0.1 g of potassium dihydrogen phosphate, 3.0 g of sucrose, and 50 mL of distilled water, and was adjusted to pH 4.1 with NaOH, and was sterilized at 100° C. for 5 minutes. The culturing medium was placed in a 250 mL beaker for static fermentation, inoculated with 5% (V/V) Glucoacetobacter xylinus, and stood and cultured at 30° C. for 6 days. A solid content of a wet bacterial cellulose pellicle obtained was 1.5 wt %.

### Embodiment 1

30 g of bacterial cellulose (BC) wet pellicle was cut into small pieces, added into 100 mL of water, separated into small fragments (2 mm in each direction) by using a tissue masher, till the small fragments were not suspended in water after stood for a period of time. The fragments were filtered, then added into 100 mL of 0.2% sodium periodate solution, and stirred at 350 rpm. A reaction was performed at a room temperature for 2 days without light. After the reaction was completed, an oxidized bacterial cellulose was filtered and washed. The oxidized bacterial cellulose was mixed with 5.6 g of polyethyleneimine and 80 mL of deionized water into a conical flask. 0.21 g of sodium cyanoborohydride was added as a catalyst. A pH of the mixture was added to 5.8 to 6 with 0.1 M hydrochloric acid. A reaction was performed under magnetic stirring at 350 rpm at a room temperature for 6 hours. After the reaction, a polyethyleneimine-modified BC was filtered and washed.

0.5 g of potassium chloropalladite (K₂PdCl₄) was dissolved in 100 ml of 70° C. hot water, and then added with the polyethyleneimine-modified BC. The mixture reacted at 70° C. for 6 hours under magnetic stirring at 350 rpm. The obtained solid product was washed with hot water and added into 100 mL of 5 mg/mL sodium borohydride solution to react at a room temperature for 1 hour, so as to reduce a supported palladium ion in situ. The obtained palladium nanoparticle-embedded BC (Pd-BC) was filtered and washed with deionized water. A mass fraction of palladium supported by the Pd-BC reached 9.7%.

The Pd-BC was mixed with bleached bagasse pulp at a mass ratio of 20% (the Pd-BC in a dry weight of paper), and uniformly dispersed with a standard paper pulp disintegrator at a consistency of 1% (m/m). Catalytic test paper was made of the mixed pulp by a standard paper handsheet former (Messmer 225, Holland). A dry weight of each sheet was controlled at 70 g/m². The paper sheet was dried at 120° C. for 20 minutes and kept away from light and air.

The catalytic test paper had a good catalytic effect on a Suzuki-Miyaura coupling reaction. 2 mmol of K₂CO₃ was used as alkali, and 16 mL of 95% ethanol, 1.1 mmol of phenylboronic acid and 1 mmol of iodobenzene were used to study a catalytic reaction which generated biphenyl at 80° C. in a 20 mL vial with a screw cap. All reactions were performed under a normal atmospheric condition without an inert gas atmosphere. After a solvent and a chemical were added, the reaction vial was closed with the cap and added into an oil bath that was preheated to 80° C. The catalytic test paper was cut into 1 cm×3 cm pieces. Four pieces of paper were used for each reaction and placed in a nylon net frame. After a temperature of the reaction vial in the oil bath reached equilibrium, the nylon frame loaded with the catalytic paper was inserted, the cap of the vial was closed to prevent air from entering, and the reaction mixture was stirred with a magnetic stirring rod. A yield of the biphenyl after the reaction for 2 hours was 99%. When the same catalytic test paper was used for 26 times, the yield thereof could still be close to 90%. In the same way, several phenylboronic acids and aryl halides with different substituents were selected, and the catalytic test paper also had a good catalytic efficiency. A catalytic reaction time and a yield were shown in Table 1 below (a reaction time and a yield of using the catalytic test paper in Embodiment 1 for generating a biphenyl product with 1 mmol of phenylboronic acid and 1.1 mmol of aryl halide by using 2.5 mmol of K₂CO₃ as an alkali in 16 mL of 95% ethanol, wherein a reaction temperature was 80° C.).

TABLE 1
[Chemical formula 1]

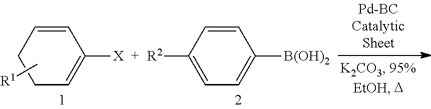

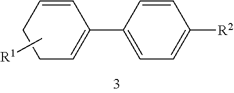

| | | | Reaction | Yield |
|---|---|---|---|---|
| X | R¹ | R² | time h | % |
| I | H | H | 1.5 | 95 |
| I | 4-COCH₃ | H | 1.5 | 100 |
| I | 4-NO₂ | H | 1.5 | 100 |
| I | 4-NH₂ | H | 3(8) | 15(80) |
| I | 4-CH₃ | H | 1.5 | 98 |
| I | 4-OCH₃ | H | 3 | 98 |
| I | H | Cl | 3 | 42 |
| I | H | OCH₃ | 3 | 36 |
| Br | H | H | 3(6) | 56(76) |
| Br | 4-COCH₃ | H | 3 | 91 |
| Br | 4-NO₂ | H | 2 | 99 |
| Br | 4-NH₂ | H | 3 | 90 |
| Br | 4-CH₃ | H | 3 | 60 |
| Br | 4-OCH₃ | H | 3(8) | 66(72) |

Note:
X = halogen;
R = substituent on benzene ring.

### Embodiment 2

30 g of bacterial cellulose (BC) wet pellicle was cut into small pieces, added into 90 mL of deionized water, separated into small fragments (2 mm in each direction) by using a tissue masher, till the small fragments were not suspended in water after stood for a period of time. The fragments were filtered, then added into 10% sodium hydroxide solution for swelling, and stirred at 350 rpm for 20 minutes. 15 mL of epoxy chloropropane was added, and then the mixture was filtered and washed after reaction for 24 hours. The epoxidized BC was added into 90 mL of deionized water, and added with 7.6 mL of tetraethylenepentamine and 1.3 g of sodium carbonate. The mixture was filtered and washed after reaction for 3 hours at a room temperature to obtain a tetraethylenepentamine-modified BC.

0.5 g of potassium chloropalladite (K₂PdCl₄) was dissolved in 100 ml of 80° C. hot water, and then added with the tetraethylenepentamine-modified BC. The mixture reacted at 80° C. for 3 hours under magnetic stirring at 350 rpm. The obtained solid product was washed with hot water and added into 100 mL of 5 mg/mL sodium borohydride solution to react at a room temperature for 1 hour, so as to reduce a supported palladium ion in situ. The obtained palladium nanoparticle-embedded BC (Pd-BC) was filtered and washed with deionized water. A mass fraction of palladium supported by the Pd-BC reached 8.2%.

The Pd-BC was mixed with bleached bagasse pulp at a mass ratio of 20% (the Pd-BC in a dry weight of paper), and uniformly dispersed with a standard paper pulp disintegrator at a consistency of 1% (m/m). Catalytic test paper was made of mixed paper pulp by a standard paper handsheet former (Messmer 225, Holland). A dry weight of each piece of paper was controlled at 70 g/m². The paper was dried at 105° C. for 30 minutes and kept away from light and air.

The catalytic test paper had a good catalytic effect on a Heck reaction and a Sonogashira reaction. 2 mmol of K₂CO₃ was used as alkali, and 16 mL of 95% ethanol, 1.1 mmol of styrene or 1.1 mmol of phenylethynyl, and 1 mmol of bromobenzene were used to study a reaction at 80° C. in a 20 mL vial with a screw cap. All reactions were performed under a normal atmospheric condition without an inert gas atmosphere. After a solvent and a chemical were added, the reaction vial was closed with the cap and added into an oil bath that was preheated to 80° C. The catalytic test paper was cut into 1 cm×3 cm pieces. Four pieces of paper were used for each reaction and placed in a nylon net frame. After a temperature of the reaction vial in the oil bath reached equilibrium, the nylon frame loaded with the catalytic paper was inserted, the cap of the vial was closed to prevent air from entering, and the reaction mixture was stirred with a magnetic stirring rod. Yields after reaction for 2 hours were 95% and 98% respectively. When the same catalytic test paper was used for 20 times, the yield thereof could still be close to 90%.

### Embodiment 3

30 g of bacterial cellulose (BC) wet pellicle was cut into small pieces, and the step was the same as that in the Embodiment 1. BC water was filtered, then added into 100 mL of pyridine, heated to 80° C., and stirred at 500 rpm for 30 minutes. After cooling to a room temperature, 10 mL of chlorodiphenyl phosphine was added to react at 350 rpm at a room temperature for 3 days. After the reaction was completed, the mixture was filtered and washed to obtain a diphenylphosphine functionalized BC. The diphenylphosphine functionalized BC was added into 100 mL of 0.2 M nickel chloride hexahydrate solution (NiCl₂.6H₂O), and stirred at 350 rpm at a room temperature for 4 hours. The solid product was filtered and washed, added into 100 mL of 0.1 M sodium cyanoborohydride solution, and stirred and reacted at a normal temperature for 1 hour to reduce a supported nickel ion in situ. An obtained nickel-nanoparticle-supported BC (Ni-BC) was filtered and washed with deionized water.

The Ni-BC was mixed with bleached softwood pulp to manufacture the catalytic test paper, and the step was the same as that in the Embodiment 1.

The catalytic test paper had a good catalytic effect on a degradation reaction of a nitro-aromatic compound. 0.8 mL of 0.2 M 2-nitrophenol solution, 1.6 mL of 0.2 M sodium borohydride solution and 10 mL of deionized water were added into a 20 mL vial with a screw cap to react at a room temperature. A usage method and a dosage of the catalytic test paper were the same as those in the Embodiment 1. A yield of 2-aminophenol after reaction for 30 minutes was 92%, When the same catalytic test paper was used for 10 times, the yield thereof could still be close to 85%.

### Embodiment 4

30 g of bacterial cellulose (BC) wet pellicle was cut into small pieces, and the step was the same as the Embodiment 1. The BC was added into a mixed solution of 100 mL of N,N-dimethylacetamide and 20 mL of thionyl chloride, and stirred at 95° C. for 3 hours to prepare a chlorinated BC. 3.28 g of N-methylimidazole was added into 100 mL of dimethyl sulfoxide (DMSO), added with the chlorinated BC after dissolution, heated to 100° C. under protection of inert gas, and stirred at 350 rpm for 12 hours. After cooling to a room temperature, the mixture was filtered and washed with acetone to obtain a N-methylimidazole-functionalized BC. The N-methylimidazole-functionalized BC was added into 100 mL of 0.1 M silver nitrate solution (AgNO₃), and reacted at 350 rpm at a room temperature for 4 hours. The obtained solid product was filtered and washed, added into 100 mL of 0.1 M sodium cyanoborohydride solution, and stirred and reacted at a normal temperature for 1 hour to reduce a supported silver ion in situ. An obtained silver-nanoparticle-supported BC (Ag-BC) was filtered and washed with deionized water.

The Ag-BC was mixed with secondary fiber wood pulp to manufacture the catalytic test paper, and the step was the same as that in the Embodiment 1.

The catalytic test paper had a good catalytic effect on a degradation reaction of a nitro-aromatic compound. 0.8 mL of 0.2 M 4-nitrophenol solution, 1.6 mL of 0.2 M sodium borohydride solution and 10 mL of deionized water were added into a 20 mL vial with a screw cap to react at a room temperature. A usage method and a dosage of the catalytic test paper were the same as those in the Embodiment 1. A yield of 4-aminophenol after reaction for 30 minutes was 95%, When the same catalytic test paper was used for 10 times, the yield thereof could still be close to 90%.

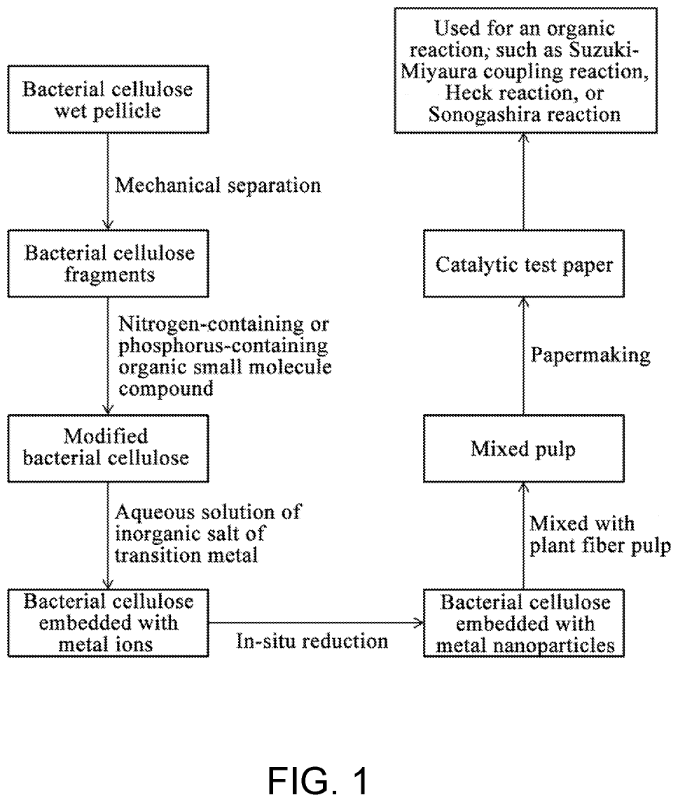

A flow chart of the present invention is shown in FIG. 1.

The embodiments enumerated above are only specific embodiments of the present invention. The present invention is not limited to the above embodiments, and may also have many variations. Any variations that are able to be directly derived from or associated with the disclosure of the present invention by those of ordinary skills in the art shall be regarded as falling within the scope of protection of the present invention.

1. A method for preparing a catalytic test paper by compositing metal particle-embedded bacterial cellulose with plant fibers, characterized in that, the method comprises the following steps:
(1) chemically bonding a nitrogen-containing or phosphorus-containing organic small molecule compound with hydroxyl groups in a structure of bacterial cellulose to obtain a functionalized bacterial cellulose having a nitrogen or phosphorus-containing group;
(2) preparing an aqueous solution of an inorganic salt of a transition metal, adding the aqueous solution into the functionalized bacterial cellulose prepared in the step (1), stirring and reacting according to a solubility of the inorganic salt of the transition metal until the nitrogen-containing or phosphorus-containing group adsorbs transition metal ions onto a nanoporous surface of the bacterial cellulose till saturation, separating and washing with water;
(3) reducing the transition metal ions adsorbed on the surface of the bacterial cellulose in the step (2) in situ to obtain bacterial cellulose embedded with transition metal nanoparticles; and
(4) mixing a plant fiber pulp with the bacterial cellulose embedded with the transition metal nanoparticles prepared in the step (3), then uniformly dispersing the mixed pulp, manufacturing the mixed pulp into a paper, and then drying the paper to an equilibrium weight to obtain the catalytic test paper.

2. The method for preparing the catalytic test paper by compositing the metal particle-embedded bacterial cellulose with the plant fibers according to claim 1, characterized in that, the bacterial cellulose in the step (1) is secreted in vitro by a bacterial microorganism, and a culturing condition is a static or dynamic fermentation culturing condition; the bacterial microorganism is one of gluconacetobacter, acetobacter, agrobacterium, pseudomonas, achromobacter, alcaligenes, aerobacter, azotobacter, rhizobium and sarcina; and the nitrogen-containing or phosphorus-containing organic small molecule compound is one or more of ethylenediamine, tetraethylenepentamine, diethylenetriamine, polyethyleneimine, N-methylimidazole and chlorodiphenyl phosphine.

3. The method for preparing the catalytic test paper by compositing the metal particle-embedded bacterial cellulose with the plant fibers according to claim 1, characterized in that, a method for bonding the nitrogen-containing or phosphorus-containing organic small molecule compound with the hydroxyl groups of the bacterial cellulose in the step (1) is to oxidize the hydroxyl groups on the bacterial cellulose into aldehyde groups in water by using an oxidant, and then bond the aldehyde groups with the nitrogen-containing compound through a reductive amination reaction; and the oxidant is one or more of a periodate or a 2,2,6,6-tetramethylpiperidine-1-oxyl radical (TEMPO) agent.

4. The method for preparing the catalytic test paper by compositing the metal particle-embedded bacterial cellulose with the plant fibers according to claim 1, characterized in that, a method for bonding the nitrogen-containing or phosphorus-containing organic small molecule compound with the hydroxyl groups of the bacterial cellulose in the step (1) is to react the bacterial cellulose with a halogen-containing epoxy compound in concentrated alkaline with a mass concentration of 10% to 20%, to bond epoxy groups with the hydroxyl groups of the bacterial cellulose, and then to react the epoxy groups with the nitrogen-containing organic small molecule compound.

5. The method for preparing the catalytic test paper by compositing the metal particle-embedded bacterial cellulose with the plant fibers according to claim 1, characterized in that, a method for bonding the nitrogen-containing or phosphorus-containing organic small molecule compound with the hydroxyl groups of the bacterial cellulose in the step (1) is to react the bacterial cellulose with thionyl chloride under dimethylformamide or N,N-dimethylacetamide, to bond chlorine atoms to the hydroxyl groups of the bacterial cellulose, and then to react with the nitrogen-containing organic small molecular compound.

6. The method for preparing the catalytic test paper by compositing the metal particle-embedded bacterial cellulose with the plant fibers according to claim 1, characterized in that, a method for bonding the nitrogen-containing or phosphorus-containing organic small molecule compound with the hydroxyl groups of the bacterial cellulose in the step (1) is to bond with the hydroxyl groups of the bacterial cellulose by using chlorodiphenyl phosphine in a condition that pyridine is used as a solvent.

7. The method for preparing the catalytic test paper by compositing the metal particle-embedded bacterial cellulose with the plant fibers according to claim 1, characterized in that, the transition metal in the step (2) is one or more of palladium, chromium, nickel, silver, copper and gold.

8. The method for preparing the catalytic test paper by compositing the metal particle-embedded bacterial cellulose with the plant fibers according to claim 1, characterized in that, a method for reducing the transition metal ions adsorbed by the bacterial cellulose in situ in the step (3) is to soak the bacterial cellulose adsorbed with the transition metal ions in a solution of a sodium borohydride, sodium cyanoborohydride or hydroxylamine hydrochloride reducing agent.

9. The method for preparing the catalytic test paper by compositing the metal particle-embedded bacterial cellulose with the plant fibers according to claim 1, characterized in that, the plant fiber pulp in the step (4) is a papermaking raw material prepared from a wood fiber, a non-wood plant fiber or a secondary fiber by a mechanical or chemical pulping method.

10. A catalytic test paper prepared by the preparation method according to claim 1.

11. A catalytic test paper prepared by the preparation method according to claim 2.

12. A catalytic test paper prepared by the preparation method according to claim 3.

13. A catalytic test paper prepared by the preparation method according to claim 4.

14. A catalytic test paper prepared by the preparation method according to claim 5.

15. A catalytic test paper prepared by the preparation method according to claim 6.

16. A catalytic test paper prepared by the preparation method according to claim 7.

17. A catalytic test paper prepared by the preparation method according to claim 8.

18. A catalytic test paper prepared by the preparation method according to claim 9.
